(19) United States
(12) Patent Application Publication
(10) Pub. No.: US 2026/0126447 A1
(43) Pub. Date: May 7, 2026

(54) METHODS OF DETECTING MICROFILARAE

(71) Applicant: Parasight System Inc., Lexington, KY (US)

(72) Inventors: Pawel Slusarewicz, Lexington, KY (US); Britt Ripley, Lexington, KY (US); Alex Chen, Lexington, KY (US); Eric Hauck, Lexington, KY (US); Kevin Calcotte, San Diego, CA (US)

(73) Assignee: Parasight System Inc., Lexington, KY (US)

(21) Appl. No.: 19/382,948

(22) Filed: Nov. 7, 2025

Related U.S. Application Data
(60) Provisional application No. 63/717,563, filed on Nov. 7, 2024.
(60) Provisional application No. 63/876,449, filed on Sep. 5, 2025.

(51) Int. Cl. G01N 33/58; G01N 1/40; G01N 21/64

(52) U.S. Cl. CPC G01N 33/582; G01N 1/4077; G01N 21/6428; G01N 21/6456; G01N 2001/4088; G01N 2021/6439

(57) ABSTRACT

Provided herein are methods for detecting the presence of microfilariae in a blood sample. Generally, the methods include pouring a blood sample comprising erythrocytes onto a porous filter, wherein the porous filter captures microfilaria on a surface of the porous filter; drawing the erythrocytes through the porous filter, leaving a remaining sample on the surface of the porous filter; fluorescently labeling the remaining sample to form a labeled sample; washing the labeled sample; imaging the surface of the porous filter using an imaging system; and detecting the presence of microfilariae based on fluorescence in the images.

### CROSS-REFERENCE TO RELATED APPLICATIONS

The present application claims priority to U.S. Provisional Ser. No. 63/717,563, filed Nov. 7, 2024, and U.S. Provisional Ser. No. 63/876,449 filed Sep. 5, 2025, the entirety of each of which is incorporated herein by reference.

### TECHNICAL FIELD

The present disclosure generally relates to methods of detecting parasitic infections in biological samples.

### BACKGROUND

Current diagnostic methods for detecting filarid infections rely on techniques such as microscopic examination, immunological, and molecular approaches, each presenting unique benefits and limitations. Microscopic examination, the simplest method, involves scanning small blood smears for the presence of microfilariae; however, its sensitivity is restricted due to the limited sample volume (about 20 μL).

Techniques that increase blood sample size, like the examination of centrifuged hematocrit tubes and specialized counting chambers, can boost detection by concentrating larvae in specific blood layers. To further enhance sensitivity, two established tests, Knott's test and membrane filtration, process larger blood volumes (up to 1 mL) by hemolyzing blood and concentrating larvae either through centrifugation or fine filtering, respectively. These methods significantly raise the likelihood of detecting low-level infections but require laborious preparation and examination. Fluorescent staining methods, including histological dyes like methylene blue and advanced options like fluoresceinated lectins, enhance visibility, while real-time PCR using fluorescence enables non-microscopic detection of filarid DNA.

Despite their effectiveness, these methods are time-consuming, labor-intensive, and require substantial laboratory expertise. Skilled personnel must carefully scan slides manually under high magnification, making the process challenging in busy clinical settings that demand prompt results. Although the natural wriggling motility of microfilariae can aid visual detection, most methods disrupt this movement due to osmotic shock, dehydration, or the use of fixatives, limiting its diagnostic utility. Consequently, these tests are often impractical for high-throughput clinical environments.

Accordingly, a need exists to develop more streamlined methods for the detection of microfilariae in biological samples without requiring extensive manual processing, thereby offering a practical solution for rapid, reliable detection.

### SUMMARY

Embodiments of the present disclosure generally relate to the detection of microfilariae in a biological sample.

In one embodiment, the present disclosure concerns a method for detecting the presence of microfilariae in a blood sample comprising: pouring a blood sample comprising erythrocytes onto a porous filter, wherein the porous filter captures microfilaria on a surface of the porous filter; drawing the erythrocytes through the porous filter, leaving a remaining sample on the surface of the porous filter; fluorescently labeling the remaining sample to form a labeled sample; washing the labeled sample; imaging the surface of the porous filter using an imaging system; and detecting the presence of microfilaria based on fluorescence in the images.

In another embodiment, the present disclosure concerns a method for detecting the presence of microfilariae in a blood sample comprising: pouring the blood sample onto a porous filter that traps the microfilariae while allowing erythrocytes to flow through; drawing the sample through the filter to remove the sample; adding fluorescent microbeads; taking video or a sequence of photographs of the sample on the filter at low magnification using a optical system tuned to detecting bead fluorescence; and using computer vision algorithms to detect bead movement caused by the motion of any microfilariae present.

It is understood that both the foregoing general description and the following detailed description are exemplary and explanatory only and are not restrictive of the present disclosure as claimed.

### BRIEF DESCRIPTION OF THE DRAWINGS

The present disclosure will be better understood from a detailed description of some example embodiments below in conjunction with the following drawings.

FIG. 1 depicts an exemplary filter assembly for filtering of blood and capture of microfilariae.

FIG. 2 depicts the movement of fluorescent beads elicited by microfilariae, according to one or more embodiments described herein. The upper panel shows successive frames from the raw video while the lower panel shows the differences between the pixels of adjacent frames following thresholding to increase the contrast.

FIG. 3 depicts direct video imaging of moving microfilariae using fluorescence optics, according to one or more embodiments described herein. Consecutive frames from a raw video are shown in the upper panel with subtle mesh distortion caused by microfilarial movement indicated by the arrows. Microfilariae become apparent following inter-frame differencing and thresholding.

FIG. 4 depicts direct video imaging of moving microfilariae using conventional white light illumination, according to one or more embodiments described herein. The image shows a single frame of a differenced video capturing the entire surface of the filter. The microfilariae can be clearly seen and are moving in the raw video.

FIG. 5 depicts ten consecutive frames of a cropped area of the video captured in FIG. 4, showing the movement of the microfilariae, according to one or more embodiments described herein.

FIG. 6 depicts automated detection of microfilariae. The raw video from FIG. 4 was processed using standard computer vision methods and the regions containing microfilariae identified. Bounding boxes of each region of interest are drawn.

FIG. 7 depicts a frame from a differenced video of microfilariae gathered using green-fluorescence optics and a green-fluorescent filter system and showing the presence of moving microfilariae, according to one or more embodiments described herein.

FIG. 8 depicts automated detection of microfilariae from a portion of the video depicted in FIG. 7.

FIG. 9 depicts a frame from a differenced video of microfilariae gathered using green-fluorescence optics showing the presence of moving microfilariae bathed in a PBS solution containing fluorescein, according to one or more embodiments described herein.

FIG. 10 depicts automated detection of microfilariae from a portion of the video depicted in FIG. 9.

FIG. 11 depicts the correlation between manual 20 μL microscopic counts of canine blood samples infected with Dirofilaria immitis with corresponding automated 1 mL counts, according to one or more embodiments described herein. The relationship was logarithmic with a high coefficient of determination (R²).

FIG. 12 depicts linearity of the correlation of counts from FIG. 9 at lower microfilariae levels. The line of best fit was determined by the least squares method and the equation of the line and coefficient of determination (R²) are shown.

FIG. 13 depicts the relative sensitivity of the manual and automated methods at very low microfilariae levels. Ten subsamples of very high dilutions (1:250 and 1:500) of D. immitis infected canine blood were analyzed manually using 20 μL blood smears as were ten 1 mL samples of the sample dilutions by the automated method, according to one or more embodiments described herein.

FIG. 14 depicts labelling of microfilariae from an infected dog blood sample under examination by fluorescence microscopy, according to one or more embodiments described herein.

FIG. 15 depicts labelling of microfilariae from an infected dog blood sample under examination by fluorescence microscopy, according to one or more embodiments described herein.

FIG. 16 depicts the effect of cysteine on image background reduction, according to one or more embodiments described herein.

FIG. 17 depicts the difference between non-infected and highly infected samples, according to one or more embodiments described herein.

FIG. 18 depicts the automated detection of microfilariae according to one or more embodiments described herein using Image J open access software.

### DETAILED DESCRIPTION

Generally, the present disclosure relates to methods of detecting the presence of parasites in a biological sample based on the motile nature of microfilariae and other parasitic larvae and/or by covalently coupling the fluorophore to the surface of the microfilariae to render them fluorescent.

Features and advantages of the present disclosure will now be described with occasional reference to specific embodiments. However, the disclosure may be embodied in different forms and should not be construed as limited to the embodiments set forth herein. Rather, these embodiments are provided so that this disclosure will be thorough and complete and will fully convey the scope of the disclosure to those skilled in the art.

Unless otherwise defined, all technical and scientific terms used herein have the same meaning as commonly understood by one of ordinary skill in the art to which the invention belongs. The terminology used in the description herein is for describing particular embodiments only and is not intended to be limiting.

The present disclosure generally relates to methods of detecting microfilaria in a biological sample. As used herein, a “biological sample” refers to any material derived from a living organism that can be used for scientific analysis or diagnostic purposes. Exemplary, non-limiting examples include blood, serum, lymphatic fluid, urine, saliva, tissue, cerebrospinal fluid, sputum, swabs, fecal samples, hair, bone marrow, combinations thereof and the like. Optionally, the biological sample is blood. Optionally, the biological sample is blood, lymph, serum, and/or plasma. In some embodiments, the biological sample is blood.

In some embodiments, the living organism is a human or veterinary subject suspected of having a parasitic infection. The subject may be a mammalian subject, optionally a human, canine (dog, wolf, coyote, etc.), feline (cat, lion, tiger, etc.), equine (horse, donkey, mule, zebra, etc.), livestock (cattle, sheep, goat, swine, etc.), mustelid (ferret, badger, otter, weasel, mink, etc.), ungulate (elk, bison, rhinoceros), and the like, though any subject suspected of having a parasitic infection is contemplated and possible. The term does not denote a particular age or sex. Thus, adult, child, and newborn subjects, as well as fetuses, whether male or female, are intended to be covered.

The parasitic infection is optionally a parasitic infection that is caused by a filarial nematode. It will be appreciated that the larval stage of filarial nematodes, microfilariae, of an infected subject may be present in the biological sample. Exemplary filarial nematodes include Dirofilaria immitis, Dirofilaria repens Wuchereria bancrofti, Brugia malayi, Brugia timori, Onchocerca volvulus, Loa loa, Setaria equine, Setaria labiatopapillosa, Onchocerca cervicalis, Elaeophora schneideri, Acanthocheilonema spp., Stephanofilaria spp., Mansonella ozzardi, mixed infections, and the like. In some embodiments, the microfilariae are D. immitis larvae.

As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. It is to be further understood that where descriptions of various embodiments use the term “comprising,” and/or “including” those skilled in the art would understand that in some specific instances, an embodiment can be alternatively described using language “consisting essentially of” or “consisting of.” The term “or a combination thereof” means a combination including at least one of the foregoing elements.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. One of ordinary skill in the art will understand that any numerical values inherently contain certain errors attributable to the measurement techniques used to ascertain the values.

It should be understood that every numerical range given throughout this specification will include every narrower numerical range that falls within such broader numerical range, as if such narrower numerical ranges were all expressly written herein. Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Ranges provided herein are understood to be shorthand for all of the values within the range. For example, a range of 1 to 25 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, or 25, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 25 may comprise 1 to 5, 1 to 10, 1 to 15, and 1 to 20 in one direction, or 25 to 20, 25 to 15, 25 to 10, and 25 to 5 in the other direction.

As used herein, the terms “improve,” “increase,” “inhibit,” “reduce,” or grammatical equivalents thereof, indicate values that are relative to a baseline or other reference measurement. In some embodiments, an appropriate reference measurement may be or comprise a measurement in a particular system (e.g., in a single subject) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

In some embodiments, the present disclosure provides methods for detecting the presence of microfilariae in a biological sample. Optionally, the biological sample is a blood sample. Generally, the method includes pouring a blood sample comprising erythrocytes onto a porous filter. As described herein, the porous filter may include pores large enough to pass erythrocytes while capturing microfilaria on a surface of the porous filter. In some embodiments, as described herein, the erythrocytes are lysed before they are able to pass through the porous filter.

Optionally, the erythrocytes are drawn through the porous filter, leaving a remaining sample on the surface of the surface of the filter. In some embodiments, the remaining sample is fluorescently labelled. As used herein, “fluorescently labelled” refers to any suitable fluorescent component added to the biological sample and/or filter, including, but not limited to, fluorescent microbeads, fluorescent dyes, fluorescent tags, quantum dots. In some embodiments, fluorescently labeling the sample includes adding a fluorophore to the sample. In some embodiments, fluorescently labeling the sample includes adding fluorescent microbeads to the sample.

Optionally, after labeling the sample, the sample is washed. In some embodiments, the labeled sample is washed using a buffer system. In some embodiments, the buffer system is an alkaline buffer system. Optionally, the buffer system does not contain amine groups. In some embodiments, the buffer system is an alkaline, non-amine containing buffer system. In some embodiments, the buffer system has a pH of about 7, about 8, about 9, about 10, about 11, or about 12. Optionally, the buffer system is a sodium bicarbonate buffer system, though any suitable buffer system is contemplated and possible. In some embodiments, the buffer system includes a reactive thiol component, such as cysteine, glutathione, mercaptosuccinic acid, thioredoxins, dithiothreitol, beta-mercaptoethanol, combinations thereof, and the like.

In some embodiments, imaging the surface of the porous filter using an imaging system and detecting the presence of microfilaria based on fluorescence in the images, described in greater detail herein.

In some embodiments, the present disclosure leverages the motility of microfilariae and other parasitic larvae, which exhibit rapid wriggling motions. This movement can serve as a surrogate diagnostic indicator or enable direct detection of the parasites. Embodiments of the present disclosure utilize imaging to capture this motion, thereby allowing for the detection of microfilariae in biological samples. Exemplary imaging techniques may include fluorescence imaging, white light imaging, video imaging, sequential still photography, single still photography, combinations thereof, and the like. In some embodiments, such as in still-imaging, subsequent images may be evaluated to detect inter-image differences caused by microfilarial movement.

As noted hereinabove, some embodiments of the present disclosure utilize fluorescence imaging to capture motion from the microfilariae. Generally, a fluorescent component is added to the biological sample and/or the mesh filter to image the sample, as described in greater detail herein. Without being bound by theory, microfilariae cause subtle distortions or refractions in the fluorescent field, making them distinguishable from other sample components under low-magnification fluorescence imaging (e.g., 0.25×-5.0×). This allows for the capture of a large field of view, minimizing the data acquisition time.. This distortion is subtle but measurable, creating a slight variation in the light's path.

In conventional static or still images, this distortion is not easily detectable due to its subtlety. Microfilariae are small and often transparent, which makes the refractive distortion minimal enough that it might be missed in a single frame of an image. Advanced computational techniques, such as pixel tracking or frame-by-frame analysis, amplify these slight distortions, enhancing the visual contrast and enabling clear identification of microfilariae based on their distinct motion patterns. In some embodiments, when video footage is captured, the slight distortions caused by the refractive properties of the microfilariae may become much more apparent. As the microfilariae move within the sample, they create dynamic changes in the way light is bent or refracted, making these changes much more noticeable in real-time motion. Video imaging, described in greater detail herein, may allow for the detection of these subtle distortions because the movement of the microfilariae over time accentuates their refractive effects on the surrounding light, which would otherwise be too subtle to detect in a still image.

In some embodiments, cells within a biological sample are tagged with a fluorescent dye. Optionally, the fluorescent dye comprises a chemically-reactive fluorescent moiety that binds to microfilaria. In some embodiments, the chemically-reactive fluorescent moiety binds to the fluorescent moiety via a covalent bond. Optionally, the chemically-reactive fluorescent moiety is added to the sample after hemolysis has been performed.

In some embodiments, the movement of the labeled microlfilaria is monitored. This movement is indicative of the presence of microfilariae and serves as a marker of parasitic infection. For example, and without being bound by theory, in some embodiments, such as when the biological sample is a blood sample, erythrocytes are tagged with fluorescent dyes. Exemplary, non-limiting fluorescent dyes include fluorescently labeled lectins, receptor-targeted agonists or antibodies, and chemically-reactive dyes like fluorescein isothiocyanate (FITC).

As noted briefly above, in some embodiments, the microfilaria are labeled by covalently binding a fluorophore to the surface of the microfilaria. Optionally, the surface of the microfilaria includes a functional group that binds with a functional group on the fluorophore to form a linker moiety that covalently binds the fluorophore to the surface of the microfilaria. Any suitable reaction mechanism is contemplated and possible. Exemplary, nonlimiting functional groups on the surface of the microfilaria may include amines, carboxyl, thiols, imidazoles, thioethers, sulfhydryls, and/or aldehydes.

In some embodiments, the functional group on the surface of the microfilaria may be a portion of an amino acid. It will be appreciated that amino acids include a central peptide bond between the amino and carboxylic acid groups, but also possess side chains. Depending on the amino acid, the side chain can be reactive, such as with arginine, histidine, lysine, aspartate, glutamate, serine, threonine, asparagine, glutamine, cysteine, methionine, and tyrosine may provide reactive functional groups that form a linker moiety with the fluorophore. Optionally, these side chains may be reacted with a number of different functional groups to link the fluorophore. These functional groups include, but are not limited to, hydrazides, alkoxyamines, isothiocyanates, isocyanates, sulphonyl chlorides, carbodiimides (e.g., 1-ethyl-3-[3-dimethylaminopropyl]carbodiimide, acyl azides, anhydrides, imido esters, N-hydroxysuccinamide esters, epoxides, fluorophenyl esters, maleimides, halo acetyls, pyridyl disulfides, and the like.

In some embodiments, the functional group on the surface of the microfilaria may be a portion of a carbohydrate, such as a carboxyl group.

With respect to the fluorophores, any suitable labeling is contemplated and possible. It will be appreciated that many such molecules are known in the art. For example, and without being bound by theory, common reactive groups include amine-reactive isothiocyanate derivatives such as FITC, amine-reactive succinimidyl esters such as NHS-fluorescein or NHS-rhodamine, and sulfhydryl-reactive maleimide-activated fluors such as Fluorescein-5-maleimide. Any suitable combination and label is contemplated and possible. Exemplary, non-limiting fluorophores include fluorescein, DAPI, Texas red, rhodamine and Alexa fluor dyes as well as other fluorescent moieties such as quantum dots.

The methods of the present disclosure may also generally include flowing a biological sample through a mesh filter to capture microfilariae on the surface of the filter before and/or during imaging. Without being bound by theory, in some embodiments, capturing the microfilariae enables large volumes of blood or other biological samples to pass through the filter, concentrating the microfilariae on the filter, thereby increasing the detection sensitivity.

It will be appreciated that the mesh filter needs to strike a balance between fluid permeability and effective microfilariae trapping. The mesh filter may be made from any suitable biocompatible material, including but not limited to metals, metal alloys, glass, polymers and the like. Optionally, the mesh filters may be woven, sintered, etched, etc. Exemplary, non-limiting mesh filters include twill or dutch weave stainless steel mesh, plain weave nylon mesh, polyethylene sulfone (PES) filters, and the like, though any suitable mesh filter is contemplated and possible.

Any suitable concentration of fluorophore is contemplated and possible. As described herein, unbound fluorophore is removed from the filter by washing. It will also be appreciated that the concentration may vary since the difference in brightness is easily compensated for by adjusting parameters on the camera such as shutter speed, aperture, and gain.

In some embodiments, the background fluorescence of the captured image can be reduced following reaction with the fluorescent label. Merely as an illustrative example, and without being bound by theory, in some embodiments, the linker moiety is formed from a reaction between an amine functional group on the surface of the microfilaria and an isothiocyanate functional group on the fluorophore. It will be appreciated that, by treating the labelled sample on the filter membrane with a solution containing a thiol to quench the fluorescence of the unreacted fluorophore., the background may be reduced. Many different thiol-containing reagents are contemplated and possible, including, but not limited to, mercaptans, dithiothreitol, cysteine and glutathione.

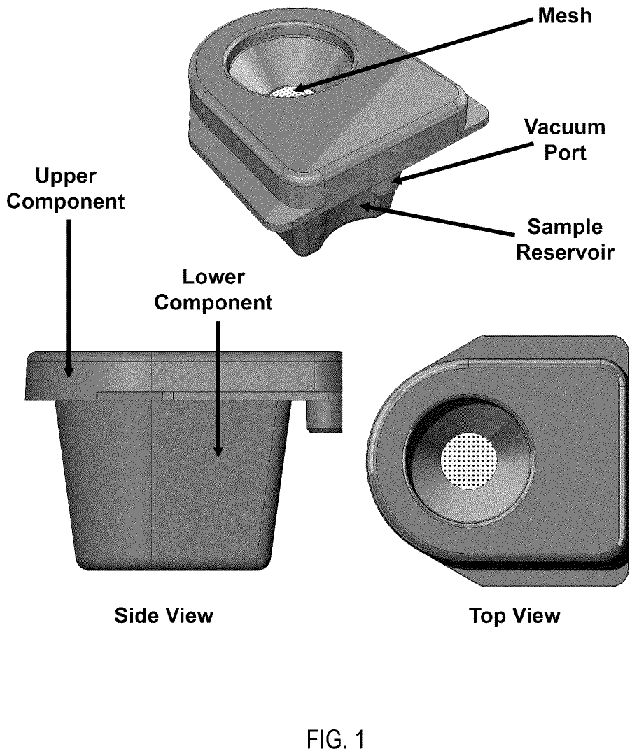

While any number of designs could be envisaged to actualize these functions, one non-limiting example is shown in FIG. 1. This filter system comprises upper and lower parts that are fused together with an air-tight seal to form a single unit. The upper half is comprised of a conical frustrum with a smaller radius of 5 mm, to which the mesh is attached, and a port to which a vacuum line can be attached. The cone serves as a receptacle into which the sample can be poured. The lower half comprises a waste receptacle into which the sample and subsequently added reagents can be drained. The mesh can be attached by any number of methods that are well known to those of ordinary skill in the art, including, but not limited to, heat staking, solvent welding and sonic welding. The top and bottom parts can be fused using similar non-limiting methods.

Applying a vacuum to the vacuum port reduces the pressure within the waste receptacle, which in turn draws through any liquids on the surface of the mesh. This facilitates the application of multiple milliliters of blood to the mesh, thereby significantly increasing the test sensitivity over other methods. Vacuum can be applied to the mesh in numerous permutations with respect to addition of the sample (and optionally beads). Multiple vacuum steps can be used to maximize the sample volume that can be drawn through the mesh. Furthermore, the reversal of the vacuum to generate upward air flow through the mesh can be used to help distribute beads evenly over the mesh surface or to pressurize the receptacle and thus maintain a water column on the mesh surface to prevent dehydration of the sample.

In some embodiments, the mesh defines pores. Optionally, the pore size is selected to ensure that only the larger structures, such as microfilariae, are retained, while smaller particles, including red blood cells, flow through the mesh filter freely. In some embodiments, the pore size of the mesh filter is selected based on the size of the microfilariae, optionally 300 microns, 250 microns, 200 microns, 150 microns, 100 microns, 90 micron, 80 microns, 75 microns, 70 microns, 60 microns, 50 microns, 40 microns, 30 microns, 25 microns, 20 microns, 15 microns, 10 microns, 5 microns etc. In some embodiments, the pore sizes are from about 2 microns to about 100 microns in diameter, including any subrange defined by any two of the aforementioned values.

In some embodiments, the biological sample is flowed through the mesh filter using passive filtration, such as gravity-driven filtration. In some embodiments, the sample is forced though the mesh by pressurizing the conical frustrum. In some embodiments, the biological sample is flowed through the mesh filter using vacuum-assisted filtration. For example, in some embodiments, a vacuum may be applied to the underside of the mesh or attached to a manifold underneath the mesh filter, accelerating the filtration process by pulling the biological sample through the mesh filter. It will be appreciated that vacuum-assisted filtration may be beneficial in applications where rapid testing and large sample throughput are required.

Optionally, the vacuum may be applied while the biological sample is being added to the mesh filter. In some embodiments, the vacuum may be applied after the biological sample has been deposited on the mesh filter, thereby removing excess fluid without disturbing any trapped microfilariae or beads. In some embodiments, the vacuum may be used with reverse airflow to distribute the fluorescent microbeads evenly and prevent dehydration of the sample. In such embodiments, the vacuum may also help ensure that beads are packed tightly enough against microfilariae, allowing their movement to be observed. In some embodiments, multiple vacuum steps can be used to maximize the sample volume that can be drawn through the mesh.

In some embodiments, the filter is washed with a non-toxic aqueous solution to remove excess blood. Optionally, the same or a different non-toxic aqueous solution may be applied to the filter prior to imaging as described in greater detail herein.

In some embodiments, for example, when the biological sample is blood, the sample is subject to hemolysis, to remove erythrocytes (which are around 5-6 microns in diameter) from the sample. It will be appreciated that, by eliminating these cells, a smaller mesh size can be used for filtering, enhancing the capacity to trap microfilariae while allowing the lysed cellular contents to pass through without clogging the filter. The hemolysis may be performed prior to placing the sample on the filter, or performed on the filter membrane itself.

In some embodiments, hemolysis is induced using ammonium chloride. Optionally, hemolysis is induced using osmotic shock. However, in some samples, osmotic shock may inhibit motion of the microfilariae, reducing their motility. It will be appreciated that this reduction in motility may make detection more difficult.

In some embodiments, the movement of the labeled erythrocytes as they are displaced by microfilariae is detected using fluorescence imaging. As the microfilariae move, they create detectable shifts in the labeled cells'position, which can be visualized in real-time and analyzed for parasite activity, described in greater detail herein. It will be appreciated that such methods may serve to streamline sample preparation. Further, the bright fluorescence provided by labeled erythrocytes may allow for lower magnification imaging, enabling rapid scanning of larger areas and/or improving processing speed. It will also be appreciated that, while fluorescence produces high-contrast images that facilitate the imaging of the microspheres, non-fluorescent, suitably dyed microspheres may also be used in conjunction with conventional illumination.

In some embodiments, motile microfilariae are imaged directly. It will be appreciated that the translucent bodies of the microfilariae allow light to refract through them, thereby causing optical distortions that may be detected as movement by the comparison of sequential images, described in greater detail herein.

In some embodiments, fluorescent microbeads are added to enable imaging of the microfilariae. Optionally, the fluorescent microbeads are added in a powder form or as a premixed solution. Generally, the fluorescent microbeads are retained on the mesh filter. In some embodiments, the fluorescent microbeads are retained on the mesh filter in a single layer that substantially covers the surface of the mesh filter. In some embodiments, the fluorescent microbeads are provided at a sufficient quantity to cover the mesh filter in a plurality of layers.

In some embodiments, the fluorescent microbeads are selected based on the size of the microbead relative to the pores in the mesh filter. It will be appreciated that the beads should be of a sufficient size to be retained by the mesh, rather than passing through it with the smaller components of the biological sample. For example, and without being bound by theory, in a 20 μm plain weave stainless steel mesh, beads larger than the mesh's pore size will be captured on its surface. The choice of mesh and bead size depends on the specific physical properties of the sample and target larvae but may also be adjusted based on the characteristics of the biological sample.

In other embodiments, the size of the beads should be small enough to be displaced by the movement of the microfilariae. When larvae wriggle or move, they push or displace the beads, causing detectable shifts in bead position. If the beads are too large, the larvae may not be able to exert enough force to move them, compromising the detection mechanism. Therefore, in some embodiments, bead size may be selected to enable responsiveness to the subtle motions of the larvae while remaining visible under fluorescence imaging. It will be appreciated that in some embodiments, the fluorescent light may be provided by a plastic component in the housing, such as the filter.

In some embodiments, the fluorescent microbeads are mixed with the biological sample. In some embodiments, the fluorescent microbeads are added to the mesh filter before the biological sample. In some embodiments, the fluorescent microbeads are added to the mesh filter simultaneously with the biological sample. In some embodiments, the fluorescent microbeads are added to the mesh filter after the addition of the biological sample.

In some embodiments, a dilute fluorophore solution, such as fluorescein, is spread across the mesh filter and/or added to the biological sample. It will be appreciated that the introduction of a fluorophore solution serves to increase the contrast between the microfilariae and the surrounding environment, making the larvae easier to detect through their interactions with the light passing through the sample, described in greater detail herein. Exemplary fluorophore solutions include, but are not limited to fluorescein (e.g., fluorescein sodium, fluorescein isothiocyanate), rhodomine, Alexa Fluor dyes, acridine orange, SYBR green, DAPI (4′,6-diamidino-2-phenylindole), calcein AM, combinations thereof, and the like. It will be appreciated that the specific fluorophore chosen may depend on factors such as the type of sample, the detection method, and the desired fluorescence characteristics (e.g., excitation/emission spectra). Furthermore, the fluorophore solution may also include ingredients that maintain the viability of the larvae (such as nutrients) or that modify the physical properties of the solution, for example viscosity to prevent dripping of the solution through the mesh under gravity.

As described herein, the embodiments of the present disclosure generally include imaging the mesh filter and/or the biological sample to detect the presence of the microfilariae. Optionally, the imaging techniques utilizes fluorescence microscopy. In some embodiments, the imaging is an automated process using computerized video motion-detection and motion-tracking algorithms. Optionally, imaging the microfilariae involves use of an automated microscopy system, optionally a fluorescence imaging system. In some embodiments, the automated microscopy system is a fluorescence imaging system that utilizes still imaging. system. In some embodiments, the automated microscopy system is a fluorescence imaging system that utilizes video imaging. In some embodiments, the automated microscopy system is a conventional light imaging system that utilizes still imaging. In some embodiments, the automated microscopy system is a conventional light imaging system that utilizes video imaging.

In some embodiments, detection by video may use fluorescence imaging or conventional light illumination, as described herein. Although fluorescence imaging may be used in circumstances where enhanced contrast is desired, the systems and methods described herein may also detect microfilariae using visible light. The video processing techniques employed are sufficiently adaptable to handle visible-light images, which provides versatility for settings where fluorescence equipment may not be available. Moreover, the imaging modality is not limited strictly to continuous video recording; the system can capture a series of consecutive still images within the same field, enabling detection of movement through localized differences between frames. This still-image approach further broadens the method's applicability, making it accessible across different types of imaging setups.

In some embodiments, the automated microscopy system includes one or more computing devices with sufficient memory and processing power to support image capture, storage, and analysis. This may include traditional computers, servers, or portable devices (e.g., tablets or smartphones) with software modules capable of image processing and data transmission. These devices are generally capable of handling high-resolution image data for detecting subtle movements or morphological features of microfilariae.

In some embodiments, imaging the microfilariae includes capturing one or more magnified images of the captured microfilariae. The magnification is generally sufficient to allow the full length of microfilariae (200-300 micrometers) to be visible within the field, optionally, from about 0.25× to about 50×, including about 0.25×, 0.5×, 0.75×, 1×, 2×, 5×, 10×, 15×, 20×, 25×, 30×, 35×, 40×, 45×, and 50×, including any subrange defined by any two of the aforementioned values. In some embodiments, phase contrast or brightfield illumination may enhance the transparency of microfilariae, distinguishing them from the background without the need for staining. Optionally, continuous video capture records the movement of the microfilaria to capture the distinctive wriggling and serpentine motions indicative of healthy larvae.

Once the images are acquired, preprocessing techniques may be employed to improve detection accuracy. Optionally, preprocessing may focus on isolating microfilariae from static background elements. Background subtraction may remove any non-motile debris or artifacts (such as blood cells). Optionally, thresholding and contrast adjustments may enhance the visibility of microfilariae's elongated forms against other particles. Noise reduction may be employed to smooth out any image artifacts, ensuring that tracking algorithms accurately follow the microfilariae's motion without interference from cellular debris. Any suitable processing method is contemplated and possible. Computation processes used in motion detection include, but are not limited to, frame-to-frame or image-to-image pixel intensity differencing, intensity thresholding, pixel erosion and/or dilation and object tracking.

The automated microscopy system may employ shape-based detection algorithms to recognize the thread-like forms of microfilariae, identifying their slightly thicker heads and tapered tails. In some embodiments, object segmentation creates binary masks that outline each microfilaria separately, labeling and preparing each one for tracking across video frames. Any suitable shape-based algorithm, with or without the use of machine-learning based image classifiers, deep-learning based approaches, such as You Only Look Once (YOLO)

Movement detection algorithms may be employed to detect the genuine wriggling motions of microfilariae, allowing for clear differentiation between live organisms and immobile artifacts. For example, in some embodiments, the automated microscopy system performs frame-by-frame tracking to map out each microfilaria's unique trajectory. Given the undulating nature of their movement, centroid tracking—focusing on the central point of each microfilaria's body—provides an efficient means of tracking the larvae. In some embodiments, optical flow analysis, which examines pixel changes to determine movement direction and speed, captures the wave-like sinusoidal patterns characteristic of microfilariae's motility. The automated microscopy system may record detailed trajectories, enabling measurement of displacement, velocity, and distinct movement patterns (e.g., linear vs. oscillatory).

In some embodiments, the automated microscopy system uses quantitative analysis capabilities to extract metrics specific to microfilarial health and activity. For example, velocity and displacement measurements may assess each microfilaria's speed and total distance traveled to evaluate infection severity and/or post-treatment activity. Amplitude and frequency of movement may provide additional indicators of vitality of the larvae, as wider and more frequent undulations are typically associated with metabolic activity. Behavioral analysis identifies periods of stillness or metabolic arrest, with software programmed to monitor shifts in movement patterns that may arise from environmental changes (such as oxygen levels or pH) or drug exposure. Population-level data, including average speed and the percentage of actively motile larvae, may provide an overview of the larvae's health, supporting infection and treatment assessments.

In some embodiments, the automated microscopy system may provide data visualization and output to offer comprehensive results, such as diagnosis, treatment efficacy, etc. For example, path visualization may overlay movement trails onto sample images, creating a visual representation of each microfilaria's trajectory. Quantitative metrics may be compiled into tables or charts, summarizing averages for speed, amplitude, and frequency. Alert triggers may notify users if microfilarial activity drops below a set threshold, which is particularly useful for viability assessments.

In some embodiments, the automated microscopy system defines automated diagnostic thresholds for movement, optionally establishing quantifiable and reproducible limits for movement parameters. In some embodiments, the automated microscopy system defines one or more parameters related to movement, including, but not limited to velocity, which refers to the average speed of movement across frames, such as the wriggling or sinusoidal motion of microfilariae; displacement, which measures the total distance traveled over time and helps assess overall motility; and amplitude and frequency, which reflect the degree of bending or undulation and the frequency of movement. Higher amplitude and frequency typically indicate more vigorous movement, suggesting healthier, viable organisms. Additionally, path curvature and oscillation patterns may serve as indicators of health, with regular, rhythmic movement suggesting viability and erratic or minimal movement potentially signaling distress or low viability.

In some embodiments, the automated microscopy system compares the sample with training data. Optionally, the training data is developed from controlled viability tests using known viable and non-viable microfilariae. In some embodiments, data is recorded under standardized conditions, including fully active (control), partially active (e.g., stressed or drug-treated), and inactive (non-viable) microfilariae. Optionally, data is collected across different environmental conditions, such as temperature, pH, and nutrient levels, to ensure that the thresholds are robust across typical diagnostic contexts. Machine learning models, such as support vector machines (SVM) or random forests, may also be used to train the system on movement data, helping identify thresholds by capturing non-linear relationships between movement parameters.

In some embodiments, the automated microscopy system defines an initial movement threshold. Optionally, the initial movement threshold is determined based on the training data. For example, a baseline velocity threshold can be defined, such as 10 microns per second, to distinguish active, viable microfilariae from non-viable ones that move slower or not at all. Low viability may be flagged using a range of movement frequencies. For example, healthy microfilariae might oscillate at 1-2 Hz, whereas stressed microfilariae may show lower frequencies, like 0.5 Hz, marking a low viability threshold.

Time-based thresholds may also be used to account for the persistence and onset of movement. The system may define the minimum active time needed for a microfilaria to be considered viable, such as 10 seconds of movement above the baseline threshold. Additionally, latency tolerance may be set, determining how quickly movement must start after sample preparation to account for slight delays in motility.

Optionally, quantitative and statistical approaches may be employed to further define threshold values. For example, threshold ratios, such as the velocity-to-displacement ratio, may help separate viable and non-viable organisms. High ratios typically suggest active, directional movement, while lower ratios indicate reduced or erratic motility.

In some embodiments, the automated microscopy system provides an automated alert when one or more parameter thresholds indicates the presence of microfilariae. Optionally, the system includes a user-friendly interface with data visualization tools. Trend graphs displaying changes in movement speed, amplitude, morphology, or fluorescence may provide a clear visual representation of treatment response. Summary statistics, such as the average reduction in speed or the percentage of viable pathogens remaining, may be presented in an accessible format for healthcare providers to quickly review. Additionally, a user interface that allows for threshold adjustments within validated limits may provide diagnostic professionals with the flexibility to fine-tune the system according to specific conditions or species. By establishing automated thresholds based on reliable metrics, the automated microscopy system may consistently and accurately identify microfilariae, enabling fast and precise diagnostics in clinical or research settings.

In some embodiments, the automated microscopy system may be designed to monitor treatment efficacy in a subject. In parasitic infections, such as filiarial nematode infections, treatment with a full dose of medication, designed to kill microfilariae, leads to a rapid death of the larvae, releasing massive amounts of toxins and increasing the risk of thromboembolism. Pairing a treatment protocol with an automated monitoring system offers a comprehensive approach to evaluating treatment efficacy, tracking patient response, and identifying complications in real time.

The integration of such a system may provide continuous and precise data, enabling timely titration adjustments and actionable insights throughout the treatment phases. In some embodiments, automated monitoring ensures that each phase of treatment—from pre-treatment evaluation to long-term follow-up—may be meticulously tracked, reducing the risk of complications and enhancing overall treatment outcomes.

After a subject has been diagnosed with a parasitic infection, baseline measurements may be established before treatment begins, based on the measured parameters of the individual subject. For example, initial measurements of relevant metrics, such as movement speed, pathogen size, count, and fluorescence levels may be used to personalize the system's thresholds for detecting changes, ensuring that subsequent measurements are compared to the subject's pre-treatment state rather than general averages. Additional markers and testing, such as flow cytometry, or blood sampling devices can quantify microfilariae presence and assess cardiovascular markers like pulmonary arterial pressure. Regular heart and lung function monitoring using automated echocardiograms and pulse oximetry may establish baseline health metrics, ensuring that any stress or abnormalities during treatment are promptly detected.

Treatment of parasitic infections generally occurs in stages. For subjects with severe infections and/or symptoms, stabilizing medications (e.g., steroids or diuretics) may be prescribed to stabilize heart or lung conditions before starting treatment. In some embodiments, an antibiotic may be administered to the tested subject for a first duration of time to target symbiotic bacteria. For example, and without being bound by theory, in a D. immitis treatment protocol, an antibiotic, such as doxycycline may be administered for 30 days to target Wolbachia bacteria that live symbiotically within heartworms. Doxycycline may be administered at a dose of approximately 10 mg/kg. In some embodiments, eliminating these bacteria weakens the parasite and/or reduces inflammation. During the antibiotic treatment phase, automated blood analysis systems may track reductions in microfilariae, for example by using the automated microscopy system, and bacterial symbionts, while sensors and other testing may monitor for adverse reactions such as systemic inflammation, ensuring the treatment's safety.

An anti-parasitic agent, such as a macrocyclic lactone, may be administered simultaneously with and/or subsequently to the antibiotic for a second duration of time to target the parasitic larvae. For example, and without being bound by theory, in a D. immitis treatment protocol, ivermectin and/or milbemycin may be administered to target microfilariae. In some embodiments, the second duration of time is the same as or longer than the first duration of time. Optionally, the dose of anti-parasitic agent may be gradually increased over the second duration of time to prevent shock-like reactions in the subject.

Throughout the administration of the anti-parasitic agent, the automated assays may measure microfilariae density in the blood, providing insight into the effectiveness of the treatment and ensuring the safe reduction of microfilariae. In some embodiments, the microfilariae density is measured at regular intervals associated with changes in the anti-parasitic dosage. In some embodiments, the automated microscopy system may also adjust dosing schedules based on real-time data to ensure gradual elimination, preventing potential shock reactions.

In some embodiments, an adulticide agent is administered to kill the adult parasites. Optionally, the adulticide is administered in stages to reduce complications, such as a two or three dose protocol. For example, and without being bound by theory, in a D. immitis treatment protocol, melarsomine dihydrochloride may be administered by injection in stages to the subject to reduce complications. In some embodiments, a three injection protocol includes a single injection of melarsomine (at about 2.5 mg/kg) to start killing adult heartworms. After 30 days, wherein the subject rests to minimize risks (e.g., pulmonary embolism), the subject receives two injections about 24 hours apart. In some embodiments, a two-dose protocol is utilized, wherein the injections are administered 24 hours apart.

Predefined diagnostic thresholds may be applied to detect responses to treatment, such as a 50% reduction in average velocity, signaling potential efficacy. To adapt over time, the system may also include dynamic threshold adjustments. As the subject's response evolves, the system may modify thresholds to avoid false positives or negatives, particularly in cases of slow or steady progress. A feedback loop may be incorporated to enable real-time treatment adjustments. By continuously monitoring treatment efficacy, the system may suggest modifications to dosing or timing. Personalized learning, through machine learning, may allow the system to recognize patterns unique to the individual and refine its understanding of what constitutes effective treatment. For example, if microfilariae viability decreases temporarily after each dose but returns to baseline before the next dose, it might indicate the need for more frequent dosing. The system could also trigger early intervention alerts if signs of treatment resistance are detected, such as no reduction in movement after a set period, prompting consideration of alternative strategies.

In some embodiments, an automated alert and reporting system would be set up to notify healthcare providers of significant changes. For instance, the system could generate alerts if there is a notable reduction in movement or a change in morphology that may indicate treatment success. Additionally, regular reports could be generated, offering visual graphs of key metrics, such as movement, pathogen count, or fluorescence intensity, to highlight trends and significant changes.

In some embodiments, post-treatment monitoring focuses on physical activity and inflammation levels. Optionally, automated sensors may track recovery by ensuring the subject adheres to rest guidelines, alerting caregivers if activity exceeds recommended thresholds. Blood-based monitors may detect markers of inflammation or hypoxia, signaling the need for corticosteroid intervention. Long-term monitoring may also include periodic automated antigen testing to confirm the elimination of heartworms and/or microfilariae screening to detect any remaining parasites, allowing for timely follow-up treatment if needed.

In some embodiments, ongoing preventative therapy is supported by continuous monitoring systems that ensure the effectiveness of monthly heartworm preventatives and/or track cardiovascular health in subjects with lingering parasitic damage. This proactive approach may help manage long-term complications, ensuring timely interventions when necessary.

While the primary application of the methods described herein are detecting filarid larvae in biological samples, its potential extends to other diagnostic and environmental contexts. For example, the methods described herein may be adapted for the detection of plant and animal parasitic nematodes in environmental samples such as soil or water.

By optimizing for speed, flexibility, and adaptability in imaging, this method provides a robust solution for tracking and identifying microfilariae and similar motile organisms across a range of sample types and environmental conditions.

A general workflow for monitoring microfilariae treatment may involve baseline assessment (measuring initial motility, count, and morphology), automated tracking (capturing images or videos at given time periods), threshold detection (flagging when motility falls below the personalized efficacy threshold), and alerts when significant milestones, like a 75% reduction in movement speed, are met. Regular progress reports, including graphs of motility and pathogen count, may help assess treatment efficacy.

By implementing such an automated, adaptive monitoring system, physicians and caregivers may track treatment efficacy dynamically, enabling timely adjustments to optimize therapeutic success.

In some embodiments, a fluorescence imaging system could instead be used to detect the microfilariae. In this case, the final aqueous solution used to cover the filter prior to imaging contains a fluorophore selected to be compatible with the light source and optical filters of the fluorescence imaging system. As a non-limiting example, the sample is illuminated with blue light with a wavelength of approximately 460 nm and the lens contains a long-pass filter with a cut-on at approximately 520 nm (which allows green and red light to pass through); in this case, fluorescein can be used as the fluorophore. Other fluorophores can also be used in conjunction with the appropriate illumination and optical filter configurations, which are well known to those of ordinary skill in the art. The light emitted by the fluorophore therefore provides the illumination necessary in the fluorescence imaging system to observe the motile microfilariae. In another aspect of the disclosure, the fluorescence is provided by a fluorophore incorporated into the material the comprises the manifold to which the filter is attached.

The embodiments described herein provide ultra-sensitive, rapid, and automated methods of detecting and treating parasitic infections in a biological sample. While the exemplified details of this disclosure are the detection of filarid larvae in animal blood, the methods disclosed herein may also be used for other purposes, including, but not limited to, the detection of plant and animal parasitic nematode larvae in environmental samples

### EXAMPLES

The general principles described herein may be used to design a large number of systems for detecting the presence and abundance of microfilaria in biological samples. For the purposes of illustration there follows a number of non-limiting examples of several assays that can be produced using these principles. The following examples are given by way of illustration and are in no way intended to limit the scope of the present disclosure.

### Example 1

A blood sample was taken from a dog with a confirmed Dirofilaria immitis infection. 70 μL of green-fluorescent microbeads (2.5 mg/mL suspension) with diameters ranging from 27 to 32 μm were added to 1 mL of the blood sample. are utilized as markers for detecting movement in the surrounding environment. The mixture was poured onto a filter system fitted with a 20 μm plain weave stainless steel mesh. The sample was aspirated by the application of vacuum and the system repressurized by reversing the air flow. Two drops (approximately 100 μL) of phosphate-buffered saline (PBS) was added to cover the filter's surface.

Five seconds of continuous video were then take at a frame rate of 3 frames/sec using filter optics compatible with fluorescein fluorescence, specifically blue LEDs attenuated with a short pass filter to attenuate any green components emitted by the LEDs and a long-pass emission filter in the lens to attenuate the blue light, at a magnification of 1× using a camera containing a 18 megapixel monochrome sensor.

FIG. 2 depicts a sequence of cropped video frames, highlighting a small section of the entire mesh of beads, focusing on the portion affected by the movement of a parasitic larva. As the larva moves through the environment, it displaces the surrounding beads. This motion is captured frame-by-frame, allowing for analysis of how the beads shift in response to the larva's activity. By tracking the movement of these fluorescent beads, researchers can indirectly observe the larva's behavior, movement patterns, and potentially even infer its level of activity or vitality, as described herein.

The upper panel depicts the raw video footage. The individual frames were then imported into photo editing software as individual layers and images of inter-frame differences were generated using the difference blending mode coupled with thresholding to enhance the differences (lower panel). The white areas in the lower panel represent regions where substantial bead movement has occurred due to the motion of a microfilaria. Automation of this process on raw video and automated detection of bead movement can be easily achieved by anyone of ordinary skill in the art of computer vision.

### Example 2

A one milliliter blood sample containing parasitic larvae, specifically microfilariae, was prepared to visually track the larvae's movement using the fluorescence-based techniques described above. To isolate and study the activity of the microfilariae within the blood, the sample was first passed through a 5 μm stainless steel mesh filter. This mesh serves as a filtration step, removing smaller blood components such as red and white blood cells while allowing larger elements, including the microfilariae, to be retained on the surface of the mesh filter.

The sample was washed three times with 200 μL of PBS After filtration of the sample, 90 μL of a dilute solution of fluorescein dye (3 μg/mL) was added to the sample to visualize changes in the environment without interfering with the visibility of the microfilariae movements.

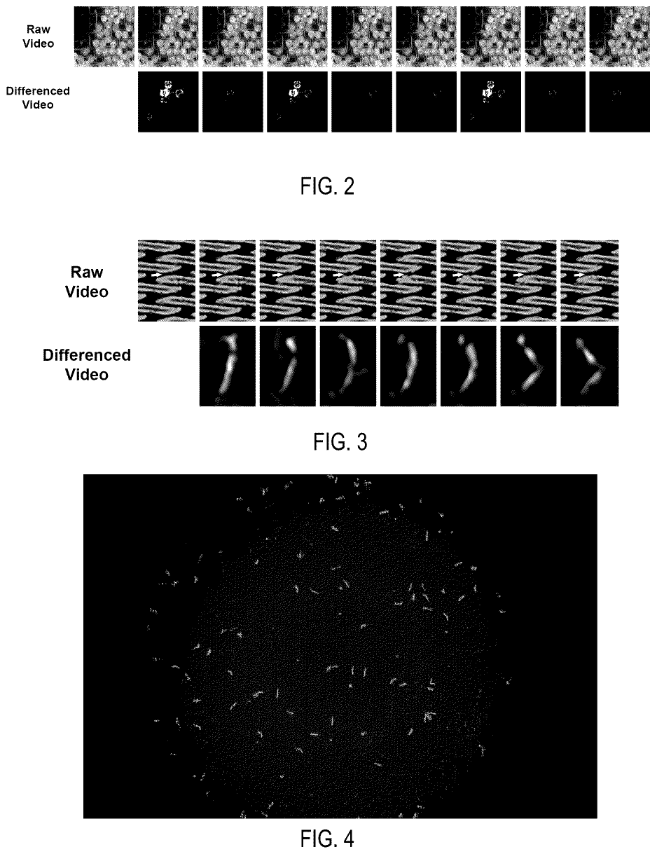

Once the sample was prepared, it was recorded on video using a microscope setup to capture fluorescein fluorescence, as described above. FIG. 3 depicts consecutive frames of the video, with arrows indicating movement of the microfilariae, allowing for detailed temporal analysis of any movements within the field of view. This movement is visualized as subtle deformations in the mesh filter. As the microfilariae move and interact with the surrounding environment, they push against or pull on the mesh filter, causing localized bending or stretching of the mesh filter.

To amplify the subtle movements captured by this system, the video was processed to highlight only pixels that differ significantly between adjacent video frames, as depicted in the lower panel of FIG. 3, although many other video processing techniques known to those of ordinary skill in the art may be used to enhance the video to highlight microfilariae movement. Movement may then be identified automatically using any of a large number of publicly available and/or proprietary motion detection algorithms.

The fluorescein solution in this example provided sufficient illumination in the fluorescence detection optical system to visualize the microfilariae movement against the background mesh. The fluorophore used does not need to be fluorescein and can instead, if need be, be selected to match the excitation and emission frequencies of the optical system. The concentration of the fluorophore can also vary substantially from the example given here, since the difference in brightness can be compensated for by adjusting parameters on the camera such as shutter speed, aperture and gain. Furthermore, the fluorophore solution can also be modified to contain ingredients that maintain the viability of the larvae (such as nutrients) or that modify the physical properties of the solution such as (as a non-limiting example) viscosity to prevent dripping of the solution through the mesh under gravity.

### Example 3

A one milliliter sample of infected blood was filtered through a 5 μm stainless steel plain Dutch weave mesh and then washed three times with 200 μL of PBS. After the addition of 90 μL of PBS the sample was then videoed as described above but using an apparatus with no optical filters and white light as an illumination source.

The resulting video was processed with an open-source computer vision software application (Open CV). The numerical values for the various processing parameters were determined empirically and will vary between imaging systems. Initially the video was processed using an implementation of the nearest-neighbors based background/foreground segmentation algorithm. This identifies moving objects within each frame by monitoring differences in the moving average values of each pixel's channels.

Detected foreground (i.e. moving) objects in each frame were masked and the frame was thresholded such that foreground pixels were white and background pixels were black. Then each frame's average intensity (white value) was evaluated, and if it exceeded a set threshold it was discarded: such frames are indicative of broad-area difference caused by sudden light level changes or camera movement. The remaining frames were summed together to form a composite image of all foreground pixels in the video.

The summed image was then denoised using OpenCV's fast non-local means denoising algorithm; this diminishes the impact of small and loosely grouped foreground pixels, and strongly reduces the influence of lone pixels that are likely to be noise. The denoised image was then dilated using OpenCV's dilation function to connect nearby points into identifiable objects.

A secondary mask was then applied to filter mesh regions known to be out of bounds and not of interest and, within the remaining region, contiguous objects/contours were identified using OpenCV's findContours function. Contours were then bounded with rectangular boxes using OpenCV's boundingRect function and contours that were too large or too small to be microfilariae were discarded to finalize the detections.

In order to produce an output where the microfilariae are more evident to the human eye, the video was processed with open-source video editing software. Such processing, however, is not critical to the practice of this disclosure since the microfilariae are detected prior to its execution. Firstly, each frame was averaged with its adjacent frames with a 1:3:1 weighting (preceding: current: following) using the “tmix” to reduce noise. The difference between each averaged frame and its following frame was computed using “tblend” filter in difference mode and each of these frames were saved as a new image series. Finally, the contrast of the final video was enhanced using the “curves” filter.

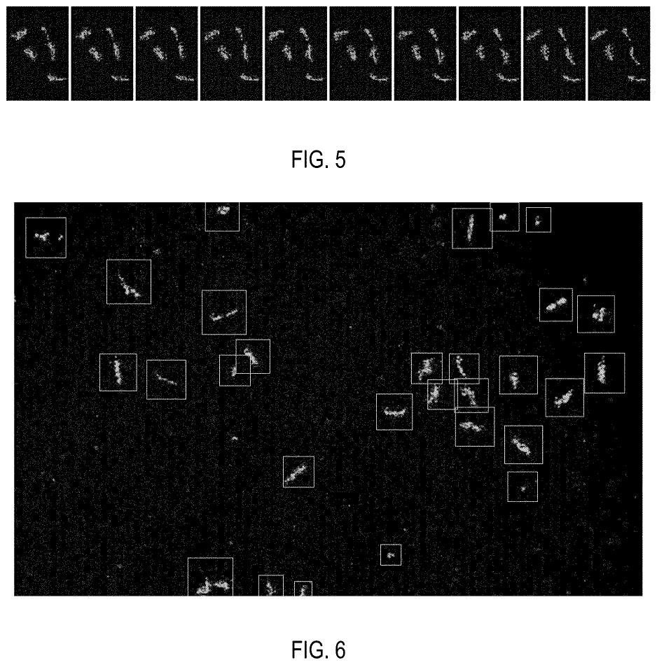

One frame from the resulting video is shown in FIG. 4, which clearly shows the position of the moving microfilariae. FIG. 5 shows 10 successive frames of a small portion of this video to highlight some of the moving larvae. FIG. 6 shows a portion of a single frame from the same video with the bounding boxes of the automated detections overlaid.

### Example 4

Microfilariae were directly imaged using the green fluorescence optics system described above and a filter system in whose housing contained 0.5% of a green-fluorescent plastic. One mL of infected blood was filtered through a 5 μm stainless steel plain Dutch weave mesh incorporated into the filter system and then washed three times with 200 μL of PBS. After the addition of 90 μL of a dilute solution of PBS the sample was videoed.

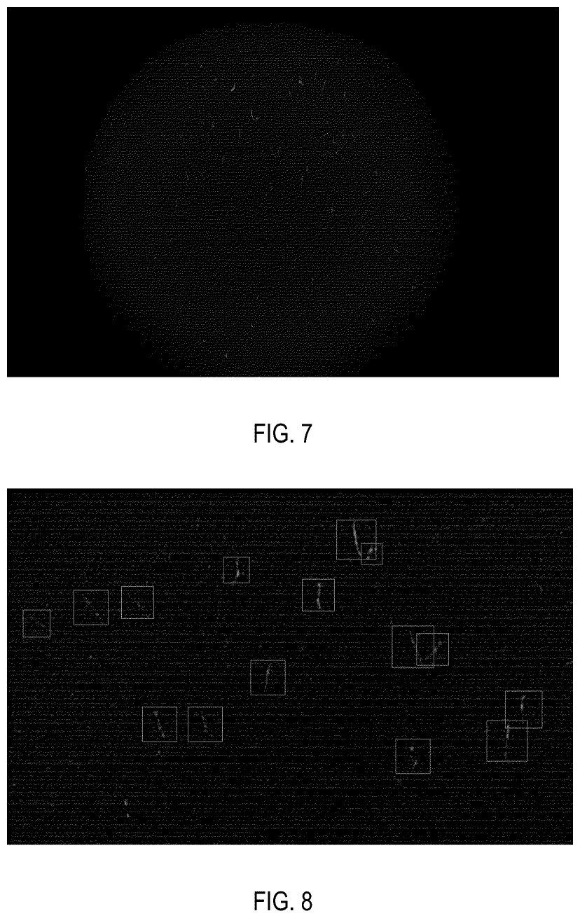

FIG. 7 shows a single frame of an output video produced by this system, where the moving larvae are clearly visible. After re-tuning OpenCV parameters to account for this illumination system, the moving larvae were detected automatically as shown in a single frame of the video depicted in FIG. 8.

### Example 5

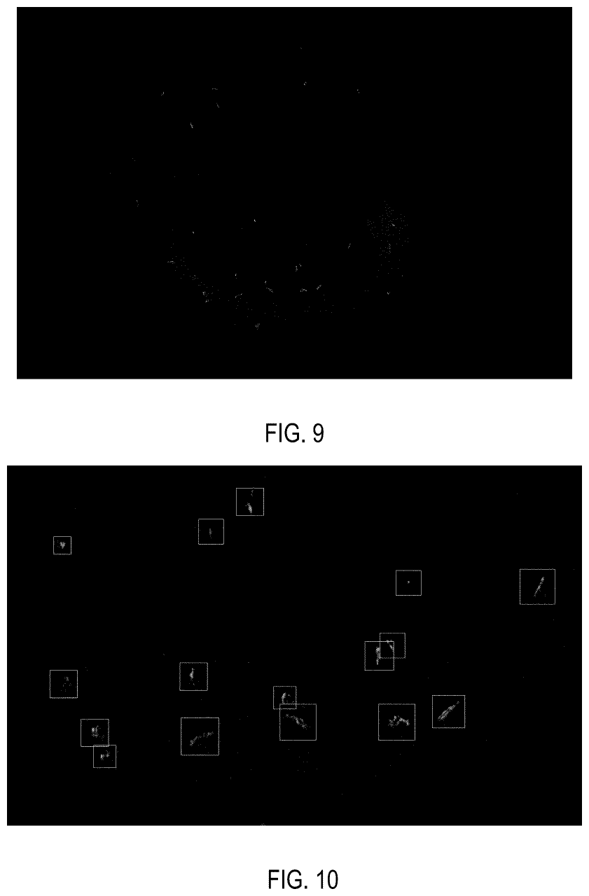

Microfilariae were directly imaged using the same fluorescence optics system using a non-fluorescent filter. One mL of infected blood was filtered through a 5 μm stainless steel plain Dutch weave mesh and then washed three times with 200 μL of PBS, followed with 200 μL of deionized water to lyse any remaining erythrocytes. After the addition of 90 μL of a dilute solution of fluorescein (3 μg/mL) in PBS the sample was videoed. FIG. 9 shows a single frame of an output video produced by this system, where the moving larvae are clearly visible. After retuning OpenCV parameters to account for this illumination system, the moving larvae were detected automatically as shown in a single frame of the video depicted in FIG. 10.

### Example 6

Microfilariae were directly imaged using the green fluorescence optics system described above and a filter system in whose housing contained 0.5% of a green-fluorescent plastic. One mL of infected blood was filtered through a 5 μm stainless steel plain Dutch weave mesh incorporated into the filter system and then washed three times with 200 μL of PBS. After the addition of 90 μL of a dilute solution of PBS the sample was videoed.

The performance of Example 5 of the disclosure was compared to a manual microscopic method commonly used in clinical veterinary practice—that is a thick smear, where approximately 20 μm of blood is overlaid with a cover slip followed by manual examination.

Infected canine blood was mixed with uninfected blood to produce serial dilutions of between 1:5 and 1:100 inclusive. Five 20 μL replicates of each dilution were examined microscopically and the number of microfilariae counted in each. Similarly, five 1 mL aliquots of each dilution were processed with the above fluorescent method and the microfilariae counted automatically as described above.

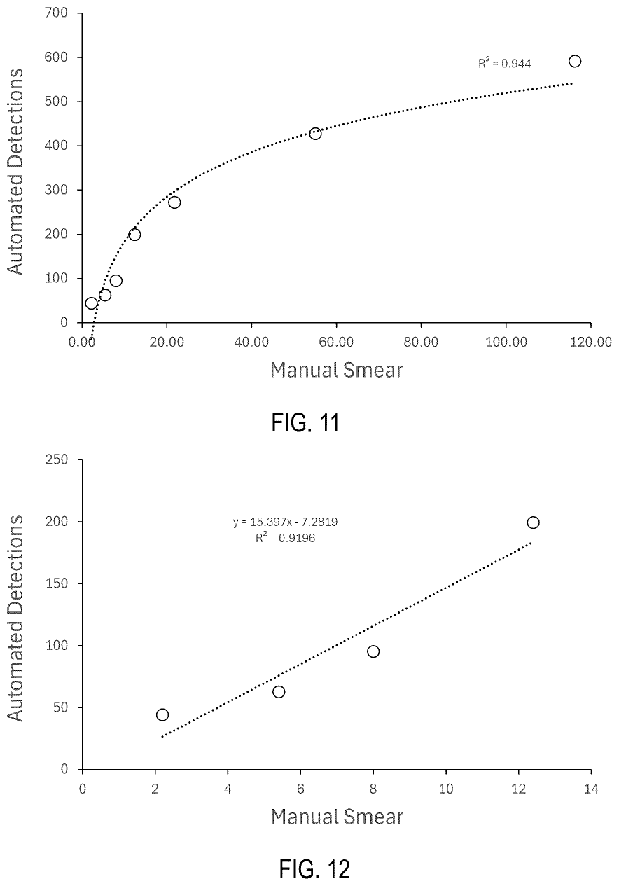

The means of each set of five manual counts was then plotted against the corresponding automated data to produce FIG. 11. There was a strong logarithmic correlation between the counts that began to plateau at higher microfilarial concentrations. This was due to the overlapping of multiple microfilariae as their concentration grew on the mesh, making them more difficult to resolve individually. At all concentrations, however, the automated method detected significantly more microfilariae than the manual blood smear.

At lower microfilariae concentrations, however, a strong linear concentration between manual and automated counts was observed, as shown in FIG. 12. In particular, the automated method was capable of detecting approximately 15 times more microfilariae than the manual (as indicated by the slope of the line in FIG. 12).

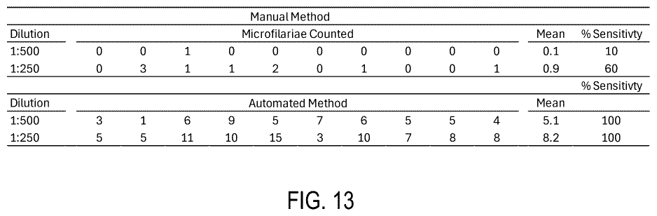

This higher number of detections resulted in significantly greater sensitivity of the automated method compared to the manual as further demonstrated in FIG. 13. Two higher dilution samples were generated that corresponded to 1:250 and 1:500 and each was counted 10 times each by both methods. At the lower concentration, nine of the ten manual counts were negative, giving a sensitivity of 10%, while at the higher concentration the sensitivity rose to 60%. In both cases, the sensitivity of the automated method was 100%, demonstrating its ability to detect the presence of microfilaria at significantly lower concentrations.

### Example 7

One milliliter of infected canine blood was filtered through a 5 μm polypropylene mesh filter. The material on the filter was then labeled by layering a solution of 5 μM fluorescein isothiocyanate (FITC) in 100 mM sodium bicarbonate buffer onto the surface of the filter and incubating at room temperature for 5 minutes. The filter was then washed twice with 100 mM sodium bicarbonate buffer, pH 10, and the material harvested from the surface with a pipette and a portion placed under a cover slip.

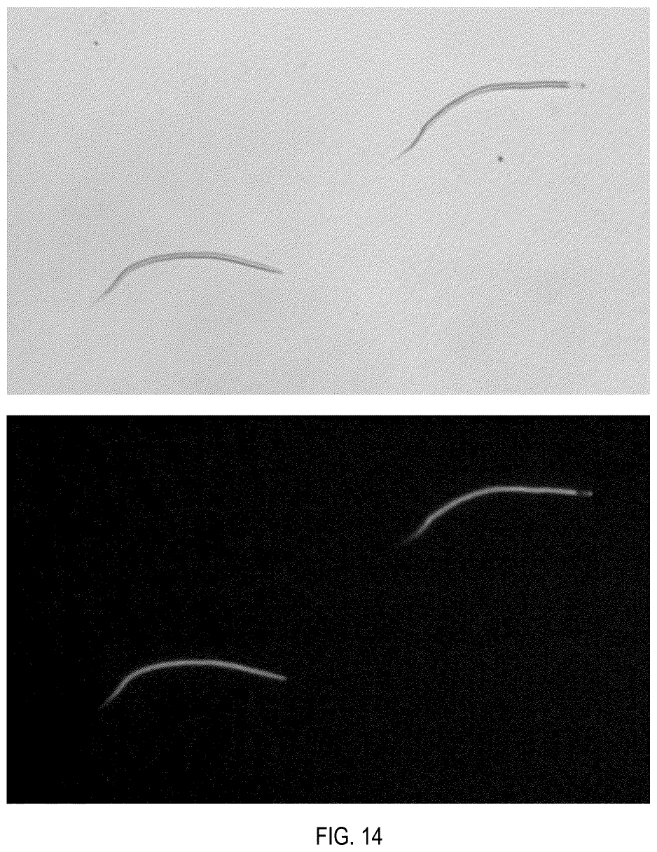

This material was then examined under a cover slip using a fluorescence microscope using standard green fluorescence optics and at 100× magnification. All the microfilariae examined (FIG. 14, upper panel) glowed intensely green (FIG. 14, lower panel).

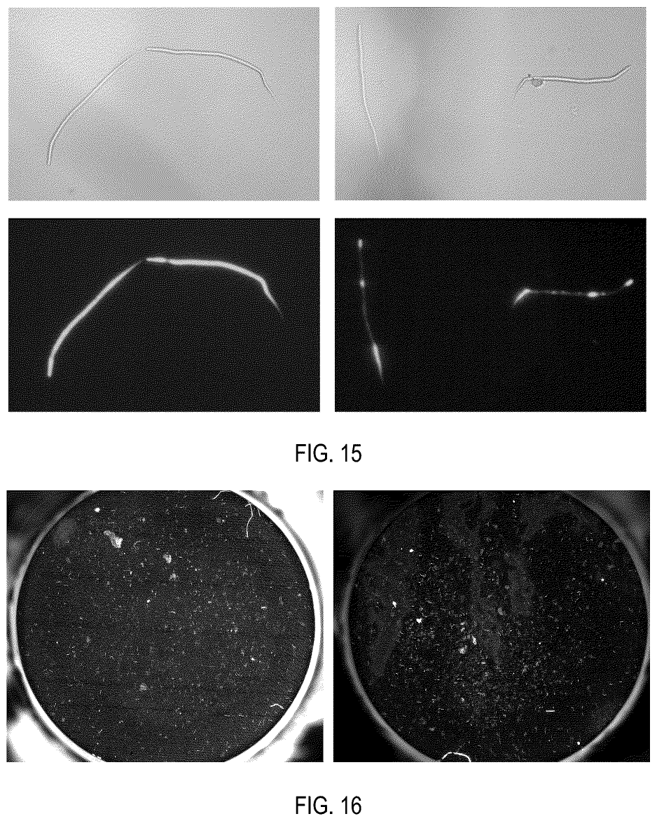

For comparison, another sample was similarly prepped but was labeled using a 5 μM solution of fluorescein maleimide in PBS. The filter was then washed with PBS. Again, all microfilariae were intensely labelled, but in this case some of the microfilariae (FIG. 15 upper panels) exhibited intense uniform staining while others stained in an intense punctate pattern (FIG. 15 lower panels). Without being bound by theory, it is contemplated that different larval stages may stain differentially with fluorescein maleimide.

### Example 8

One milliliter of infected blood containing low levels of microfilariae (approximately 500 /ml) was diluted with 3 ml of deionized water. The solution was then filtered through a 5 μm polypropylene mesh filter. The material on the filter was then treated with 1 μM FITC in 100 mM bicarbonate buffer, pH 10, and incubated at room temperature for 30 seconds. The filter was then washed 4 times with the bicarbonate buffer. The sample was imaged at 1× magnification to capture the entire mesh using the fluorescence optics described above, shown in FIG. 16 (left panel).

A second 1 mL of the same sample containing low levels of microfilariae (approximately 500 /ml) was diluted with 3 ml of deionized water. The solution was then filtered through a 5 μm polypropylene mesh filter. The material on the filter was then treated with 1 μM FITC in 100 mM bicarbonate buffer, pH 10, and incubated at room temperature for 30 seconds. The filter was then washed 4 times with the bicarbonate buffer supplemented with 100 mM cysteine. The sample was imaged at 1× magnification to capture the entire mesh using the fluorescence optics described above, shown in FIG. 16 (right panel).

As shown in FIG. 16, microfilariae were visible in both images, however the background in the sample washed in the absence of cysteine (FIG. 16, left panel) was substantially higher than that washed in its presence (FIG. 16, right panel).

### Example 9

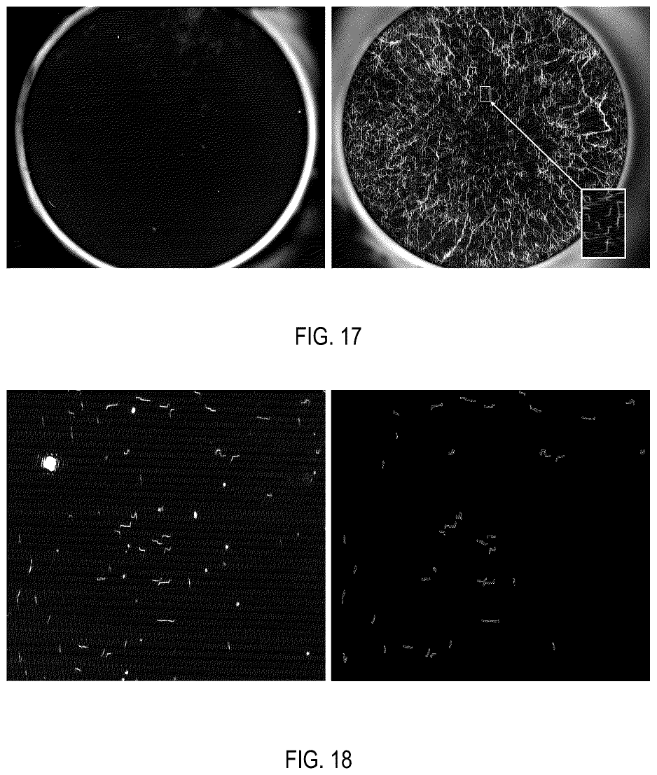

One milliliter of uninfected blood was diluted with 1 ml of deionized water. The solution was then filtered through a 5 μm polypropylene mesh filter. The material on the filter was then treated with 3 μM FITC in acetonitrile. The sample was incubated at room temperature for 10 seconds. The filter was then washed 4 times with the 100 mM bicarbonate buffer, pH 10 buffer, supplemented with 100 mM cysteine. The sample was imaged at 1× magnification to capture the entire mesh using the fluorescence optics described above. This is shown in FIG. 17, left panel.

One milliliter of highly-infected blood (approximately 50,000 microfilariae/mL) was diluted with 1 ml of deionized water. The solution was then filtered through a 5 μm polypropylene mesh filter. The material on the filter was then treated with 3 μM FITC in acetonitrile. The sample was incubated at room temperature for 10 seconds. The filter was then washed 4 times with the 100 mM bicarbonate buffer, pH 10 buffer, supplemented with 100 mM cysteine. The sample was imaged at 1× magnification to capture the entire mesh using the fluorescence optics described above. This is shown in FIG. 17, right panel As shown in FIG. 17, the uninfected sample (left panel) contained only particles and large fibers that could not be mistaken for microfilariae. The highly-infected blood sample (right panel) was covered with microfilariae to such an extent that the microfilariae had twisted together to from rope-like structures (right panel, inset.)

### Example 10

One milliliter of infected blood containing low levels of microfilariae (approximately 500 /ml) was diluted with 1 ml of deionized water. The solution was then filtered through a 5 μm polypropylene mesh filter. The material on the filter was then treated with 3 μM FITC in acetonitrile. The sample was incubated at room temperature for 10 seconds. The filter was then washed 4 times with the 100 mM bicarbonate buffer, pH 10 buffer, supplemented with 100 mM cysteine. The sample was imaged at 1× magnification to capture the entire mesh using the fluorescence optics described above. This is shown in FIG. 18, left panel.

The resulting image was then processed with Image J public open source software. After thresholding to remove the duller components of the image, including the underlying mesh, the remaining particles were analyzed and filtered based on surface area and circularity. This relatively crude image analysis method was capable of detecting the majority of the microfilariae in the image (FIG. 18, right panel) while ignoring all the non-parasitic debris on the mesh surface The methods for detecting microfilariae according to the present disclosure may also be described with reference to the following numbered clauses:

1. A method for detecting the presence of microfilariae in a blood sample comprising: pouring a blood sample comprising erythrocytes onto a porous filter, wherein the porous filter captures microfilaria on a surface of the porous filter; drawing the erythrocytes through the porous filter, leaving a remaining sample on the surface of the porous filter; fluorescently labeling the remaining sample to form a labeled sample; washing the labeled sample; imaging the surface of the porous filter using an imaging system; and detecting the presence of microfilaria based on fluorescence in the images.

2. The method of any other clause provided herein, wherein fluorescently labeling the sample comprises adding a fluorophore.

3. The method of any other clause provided herein, wherein the fluorophore is selected from fluorescein, DAPI, Texas red, rhodamine and Alexa fluor dyes.

4. The method of any other clause provided herein, wherein fluorescently labeling the sample comprises adding fluorescent microbeads.

5. The method of any other clause provided herein, wherein washing the labeled sample comprises washing with a buffer system, optionally an alkaline, non-amine buffer system.

6. The method of any other clause provided herein wherein the buffer system is a sodium bicarbonate buffer system.

7. The method of any other clause provided herein, wherein the buffer system comprises a thiol-containing component.

8. The method of any other clause provided herein, wherein the thiol-containing component is cysteine.

9. The method of any other clause provided herein, wherein the blood sample is lysed prior to pouring onto the filter.

10. The method of any other clause provided herein, wherein the sample on the filter is lysed prior to imaging.

11. The method of any other clause provided herein, wherein the sample is imaged using mono-or poly-chromatic light with no optical filtration.

12. The method of any other clause provided herein, wherein magnification of the imaging system is between 0.25× and 10×.

13. A method for detecting the presence of microfilariae in a blood sample comprising: pouring the blood sample onto a porous filter that traps the microfilariae while allowing erythrocytes to flow through; drawing the sample through the filter to remove the sample; adding fluorescent microbeads; taking video or a sequence of photographs of the sample on the filter at low magnification using a optical system tuned to detecting bead fluorescence; and using computer vision algorithms to detect bead movement caused by the motion of any microfilariae present.

14. The method of any other clause provided herein wherein the sample on the filter is washed before imaging.

15. The method of any other clause provided herein, wherein the diameter of the microbeads is between 10 and 60 μm.

16. The method of any other clause provided herein, wherein the diameter of the microbeads is between 20 and 40 μm.

17. The method of any other clause provided herein, wherein the fluorescent microbeads are added to the blood sample prior to pouring the blood sample onto the filter.

18. The method of any other clause provided herein, wherein the fluorescent microbeads are added directly to the porous filter.

19. The method of any other clause provided herein, wherein the blood sample is poured onto the porous filter subsequent to the fluorescent microbeads.

20. The method of any other clause provided herein, wherein the fluorescent microbeads are added to the porous filter subsequent to the blood sample being poured.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross-referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method for detecting the presence of microfilariae in a blood sample comprising:
pouring a blood sample comprising erythrocytes onto a porous filter, wherein the porous filter captures microfilaria on a surface of the porous filter;
drawing the erythrocytes through the porous filter, leaving a remaining sample on the surface of the porous filter;
fluorescently labeling the remaining sample to form a labeled sample;
washing the labeled sample;
imaging the surface of the porous filter using an imaging system; and
detecting the presence of microfilaria based on fluorescence in the images.

2. The method of claim 1, wherein fluorescently labeling the sample comprises adding a fluorophore.

3. The method of claim 1, wherein the fluorophore is selected from fluorescein, DAPI, Texas red, rhodamine and Alexa fluor dyes.

4. The method of claim 1, wherein fluorescently labeling the sample comprises adding fluorescent microbeads.

5. The method of claim 1, wherein washing the labeled sample comprises washing with an alkaline, non-amine buffer system.

6. The method of claim 5, wherein the buffer system is a sodium bicarbonate buffer system.

7. The method of claim 5, wherein the buffer system comprises a thiol-containing component.

8. The method of claim 7, wherein the thiol-containing component is cysteine.

9. The method of claim 1, wherein the blood sample is lysed prior to pouring onto the filter.

10. The method of claim 1, wherein the sample on the filter is lysed prior to imaging.

11. The method of claim 1, wherein the sample is imaged using mono-or poly-chromatic light with no optical filtration.

12. The method of claim 1, wherein magnification of the imaging system is between 0.25× and 10×.

13. A method for detecting the presence of microfilariae in a blood sample comprising:
pouring the blood sample onto a porous filter that traps the microfilariae while allowing erythrocytes to flow through;
drawing the sample through the filter to remove the sample;
adding fluorescent microbeads;
taking video or a sequence of photographs of the sample on the filter at low magnification using a optical system tuned to detecting bead fluorescence;
and using computer vision algorithms to detect bead movement caused by the motion of any microfilariae present.

14. The method of claim 13, wherein the sample on the filter is washed before imaging.

15. The method of claim 13, wherein the diameter of the microbeads is between 10 and 60 μm.

16. The method of claim 13, wherein the diameter of the microbeads is between 20 and 40 μm.

17. The method of claim 13, wherein the fluorescent microbeads are added to the blood sample prior to pouring the blood sample onto the filter.

18. The method of claim 13, wherein the fluorescent microbeads are added directly to the porous filter.

19. The method of claim 7, wherein the blood sample is poured onto the porous filter subsequent to the fluorescent microbeads.

20. The method of claim 13, wherein the fluorescent microbeads are added to the porous filter subsequent to the blood sample being poured.
